(19) United States
(12) Patent Application Publication
(10) Pub. No.: US 2024/0325074 A1
(43) Pub. Date: Oct. 3, 2024

(54) PASSIVE STEERABLE DILATOR

(71) Applicant: Boston Scientific Scimed, Inc., Maple Grove, MN (US)

(72) Inventors: Neal Eidenschink, Wayzata, MN (US); Nicholas Lee Tassoni, Andover, MN (US)

(21) Appl. No.: 18/615,371

(22) Filed: Mar. 25, 2024

Related U.S. Application Data
(60) Provisional application No. 63/492,422, filed on Mar. 27, 2023.

(51) Int. Cl. A61B 18/14; A61B 17/00; A61B 18/00; A61B 90/00; A61M 25/00; A61M 25/06

(52) U.S. Cl. CPC A61B 18/1492; A61B 2017/00247; A61B 2017/00336; A61B 2017/00407; A61B 2018/00357; A61B 2018/00601; A61B 2018/00946; A61B 2090/3966; A61M 25/0097; A61M 2025/0681

(57) ABSTRACT

A system for use in a transseptal crossing procedure includes an outer cannula having a proximal end, a tapered distal end, a preset curve, and a proximal portion extending between the proximal end and the preset curve. An inner member is configured to slide within the outer cannula between a first position and a second position, the inner member has a stiffness greater than a stiffness of the outer cannula. In the first position the tapered distal end is positioned approximately 90 degrees relative to the proximal portion and in the second position the tapered distal end is positioned greater than 90 degrees relative to the proximal portion. A control mechanism includes a first portion connected to the outer cannula and a second portion connected to the inner member, and is configured to move the inner member between the first position and the second position.

### CROSS REFERENCE TO RELATED APPLICATIONS

This application claims the benefit of U.S. Provisional Application No. 63/492,422 entitled “PASSIVE STEERABLE DILATOR,” filed Mar. 27, 2023, which is incorporated by reference in its entirety.

### TECHNICAL FIELD

The present invention relates generally to methods and devices usable to within the body of a patient. More specifically, the present invention is concerned with an apparatus for performing a transseptal crossing procedure.

### BACKGROUND

Devices currently exist for creating a puncture, channel, or perforation within a tissue located in a body of a patient. One particular medical procedure of interest is a transseptal crossing procedure to provide access to the left atrium of the heart, whereby a physician may use a sheath and dilator to support a crossing, puncturing, or piercing device. In such procedures, the puncturing device creates an initial perforation through the atrial septum, and the dilator is advanced across the septal puncture to enlarge the opening so that larger-diameter devices, e.g., a delivery sheath or therapy device, may be advanced into the left atrium. There is a continuing need for improved devices and methods to facilitate left atrial access.

### SUMMARY

One aspect of the disclosure is directed to a system for use in a transseptal crossing procedure. The system includes an outer cannula having a proximal end, a tapered distal end, a preset curve, and a proximal portion extending between the proximal end and the preset curve. The preset curve is configured to position the tapered distal end at approximately 90 degrees relative to the proximal portion.

The system includes an inner member configured to slide within the outer cannula between a first position and a second position, the inner member having a bend rigidity greater than a bend rigidity of the outer cannula. In the first position the tapered distal end is positioned approximately 90 degrees relative to the proximal portion and in the second position the tapered distal end is positioned greater than 90 degrees relative to the proximal portion.

The system includes a control mechanism having a first portion connected to the proximal end of the outer cannula and a second portion connected to a proximal end of the inner member. The control mechanism is configured to move the inner member between the first position and the second position.

The system includes a piercing member. In some aspects, the piercing member includes a distal radiofrequency electrode.

In some aspects, the control mechanism comprises a handle.

In some aspects, the control mechanism comprises a slider.

In some aspects, the slider forms part of a ratcheting system configured to lock the inner member in a desired position within the outer cannula.

In some aspects, the slider moves along a track that has a length substantially the same as an arc length of the preset curve.

In some aspects, the outer cannula is formed of one or more polymer and metallic material.

In some aspects, one or more marker is located on a portion of the outer cannula or the inner member.

In some aspects, the one or more marker is radiopaque.

In some aspects, the outer cannula or the inner member includes a reinforcing material.

In some aspects, the outer cannula has a bend rigidity in the range of 0.01 to 4.62 pounds-force per square inch (lbf/in²) and the inner member has a stiffness in the range of 0.07 to 130.42 lbf/in².

In some aspects, the inner member includes a beveled or polished distal tip.

In some aspects, the inner member includes one or more slot or groove and increases in flexibility from a proximal end to a distal end.

Another aspect of the disclosure is directed to dilator for use in a transseptal crossing procedure. The dilator includes an outer cannula having a proximal end, a tapered distal end, a preset curve, and a proximal portion extending between the proximal end and the preset curve.

The dilator includes an inner member configured to slide within the outer cannula between a first position and a second position. The inner member has a stiffness greater than a stiffness of the outer cannula. In the first position the tapered distal end is positioned approximately 90 degrees relative to the proximal portion and in the second position the tapered distal end is positioned greater than 90 degrees relative to the proximal portion.

The dilator includes a control mechanism having a first portion connected to the proximal end of the outer cannula and a second portion connected to a proximal end of the inner member. The control mechanism is configured to move the inner member between the first position and the second position.

In some aspects, the preset curve has an arc length in the range of 1 to 8 inches.

In some aspects, the preset curve has a radius of curvature in the range of 2 to 5 inches.

Another aspect of the disclosure is directed to a method for performing a transseptal procedure. The method includes positioning an outer cannula having an inner stiffening member into a right atrium of a patient. The method includes withdrawing the inner stiffening member from a portion of the outer cannula to change a shape of the outer cannula. A distal end of the outer cannula is placed on the transseptal septum, and a piercing member is advanced though the inner stiffening member and the outer cannula. The transseptal septum is then punctured with the piercing member.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

Example 1 is a system for use in a transseptal crossing, comprising:

an outer cannula having a proximal end, a tapered distal end, a preset curve, and a proximal portion extending between the proximal end and the preset curve, wherein the preset curve is configured to position the tapered distal end at approximately 90 degrees relative to the proximal portion; and an inner member configured to slide within the outer cannula between a first position and a second position, the inner member having a stiffness greater than a stiffness of the outer cannula, wherein in the first position the tapered distal end is positioned approximately 90 degrees relative to the proximal portion and in the second position the tapered distal end is positioned greater than 90 degrees relative to the proximal portion.

Example 2 is the system of Example 1, further comprising a control mechanism having a first portion connected to the proximal end of the outer cannula and a second portion connected to a proximal end of the inner member, wherein the control mechanism is configured to move the inner member between the first position and the second position.

Example 3 is the system of any of Examples 1 or 2, further comprising a piercing member.

Example 4 is the system of Example 3, wherein the piercing member includes a distal radiofrequency electrode.

Example 5 is the system of any of Examples 2-4, wherein the control mechanism comprises a handle.

Example 6 is the system of any of Examples 2-5, wherein the control mechanism comprises a slider.

Example 7 is the system of Example 6, wherein the slider forms part of a ratcheting system configured to lock the inner member in a desired position within the outer cannula.

Example 8 is the system of any of Examples 6 or 7, wherein the slider moves along a track.

Example 9 is the system of Example 8, wherein the track has a length that is substantially the same as an arc length of the preset curve.

Example 10 is the system of any of Examples 1-9, wherein the outer cannula is formed of one or more polymer and metallic material.

Example 11 is the system of any of Examples 1-10, further comprising one or more marker located on a portion of the outer cannula or the inner member.

Example 12 is the system of Example 11, wherein the one or more marker is radiopaque.

Example 13 is the system of any of Examples 1-12, wherein the outer cannula or the inner member includes a reinforcing material.

Example 14 is the system of any of Examples 1-13, wherein the outer cannula has a bend rigidity in the range of 0.01 to 4362 lbf/in² and the inner member has a bend rigidity in the range of 0.07 to 130.42 lbf/in².

Example 15 is the system of any of Examples 1-14, wherein the inner member includes a beveled or polished distal tip.

Example 16 is a system for use in a transseptal crossing, comprising: an outer cannula having a proximal end, a tapered distal end, a preset curve, and a proximal portion extending between the proximal end and the preset curve, wherein the preset curve is configured to position the tapered distal end at approximately 90 degrees relative to the proximal portion; an inner member configured to slide within the outer cannula between a first position and a second position, the inner member having a stiffness greater than a stiffness of the outer cannula, wherein in the first position the tapered distal end is positioned approximately 90 degrees relative to the proximal portion and in the second position the tapered distal end is positioned greater than 90 degrees relative to the proximal portion; and a control mechanism having a first portion connected to the proximal end of the outer cannula and a second portion connected to a proximal end of the inner member, wherein the control mechanism is configured to move the inner member between the first position and the second position.

Example 17 is the system of Example 16, further comprising a piercing member.

Example 18 is the system of Example 17, wherein the piercing member includes a distal radiofrequency electrode.

Example 19 is the system of Example 16, wherein the control mechanism comprises a handle.

Example 20 is the system of Example 16, wherein the control mechanism comprises a slider.

Example 21 is the system of Example 20, wherein the slider forms part of a ratcheting system configured to lock the inner member in a desired position within the outer cannula.

Example 22 is the system of Example 20, wherein the slider moves along a track.

Example 23 is the system of Example 22, wherein the track has a length that is substantially the same as an arc length of the preset curve.

Example 24 is the system of Example 16, wherein the outer cannula is formed of one or more polymer and metallic material.

Example 25 is the system of Example 16, further comprising one or more marker located on a portion of the outer cannula or the inner member.

Example 26 is the system of Example 25, wherein the one or more marker is radiopaque.

Example 27 is the system of Example 16, wherein the outer cannula or the inner member includes a reinforcing material.

Example 28 is the system of Example 16, wherein the outer cannula has a bend rigidity in the range of 0.01 to 4.62 lbf/in² and a modulus of elasticity in the range of 1×10⁷ to 1×10⁹ Pa.

Example 29 is the system of Example 16, wherein the inner member has a bend rigidity in the range of 0.07 to 130.42 lbf/in² and a modulus of elasticity in the range of 3×10⁹ to 5×10¹¹ Pa.

Example 30 is the system of Example 16, wherein the inner member includes a beveled or polished distal tip.

Example 31 is the system of Example 16, wherein the inner member includes one or more slot or groove and increases in flexibility from a proximal end to a distal end.

Example 32 a dilator for use in a transseptal crossing, comprising: an outer cannula having a proximal end, a tapered distal end, a preset curve, and a proximal portion extending between the proximal end and the preset curve; an inner member configured to slide within the outer cannula between a first position and a second position, the inner member having a stiffness greater than a stiffness of the outer cannula, wherein in the first position the tapered distal end is positioned approximately 90 degrees relative to the proximal portion and in the second position the tapered distal end is positioned greater than 90 degrees relative to the proximal portion; and a control mechanism having a first portion connected to the proximal end of the outer cannula and a second portion connected to a proximal end of the inner member, wherein the control mechanism is configured to move the inner member between the first position and the second position.

Example 33 is the dilator of Example 32, wherein the preset curve has an arc length in the range of 1 to 8 inches.

Example 34 is the dilator of Example 32, wherein the preset curve has a radius of curvature in the range of 2 to 5 inches.

Example 35 is a method for performing a transseptal procedure, comprising: positioning an outer cannula having an inner stiffening member into a right atrium of a patient; withdrawing the inner stiffening member from a portion of the outer cannula to change a shape of the outer cannula; placing a distal end of the outer cannula on the transseptal septum; advancing a piercing member though the inner stiffening member and the outer cannula; and puncturing the transseptal septum with the piercing member.

### BRIEF DESCRIPTION OF THE DRAWINGS

FIGS. 1A-1C are schematic illustrations of a medical procedure within a patient's heart utilizing a transseptal access system according to embodiments of the disclosure.

FIG. 2 illustrates a system for transseptal access having an inner member retracted to a most proximal position.

FIG. 3 illustrates the system for transseptal access in FIG. 2 having the inner member advanced to a most distal position.

FIG. 4A illustrates a perspective view of mechanism for advancing and retracting an inner member.

FIG. 4B illustrates a partial cross-sectional view of the mechanism for advancing and retracting an inner member.

FIG. 5 illustrates a plurality of curves that can be achieved by moving the inner member between a most proximal position and a most distal position.

FIG. 6A illustrates an example configuration for an inner member.

FIG. 6B illustrates an example configuration for a distal tip of an inner member.

FIG. 7 illustrates the system of FIG. 2 in a first configuration during a portion of a transseptal access procedure.

FIG. 8 illustrates the system of FIG. 2 in a second configuration during a portion of a transseptal access procedure.

FIG. 9 illustrates another embodiment of a transseptal access system including a pre-curved inner member and a straight outer cannula.

FIG. 10 illustrates another embodiment for transseptal access system including a pre-curved outer cannula and a pre-curved inner member.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

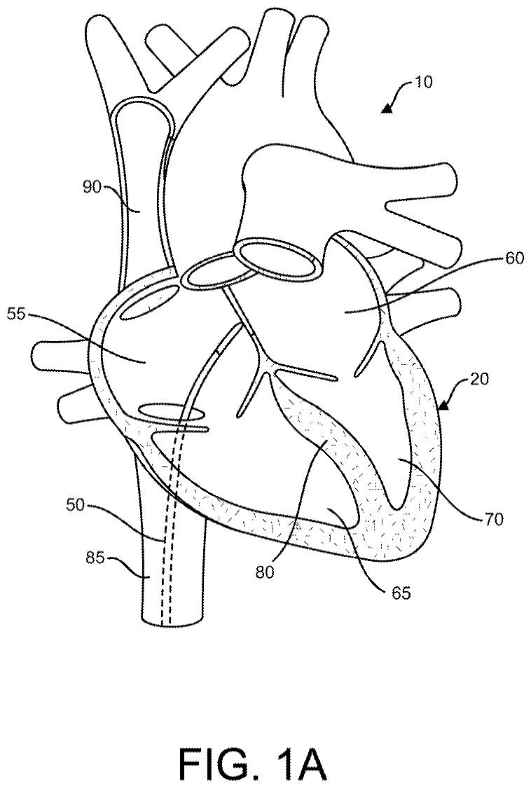

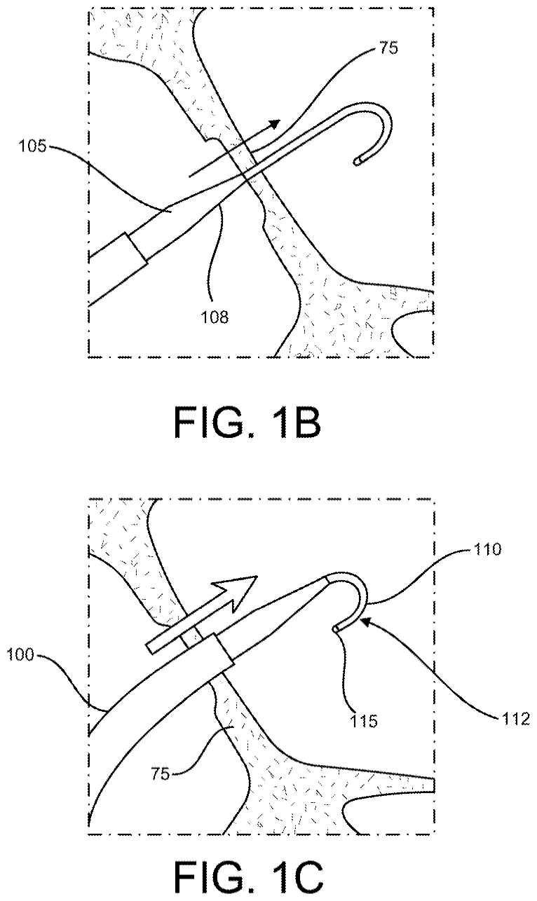

FIGS. 1A-1C are schematic illustrations of a medical procedure 10 within a patient's heart 20 utilizing a transseptal access system 50 according to embodiments of the disclosure. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 10 illustrated in FIGS. 1A-1C is an exemplary embodiment for providing access to the left atrium 60 using the transseptal access system 50 for subsequent deployment of the aforementioned diagnostic and/or therapeutic devices within the left atrium 60. As shown in FIGS. 1A-1C, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, the transseptal access system 50 includes an introducer sheath 100, a dilator 105 having a dilator body 107 and a tapered distal tip portion 108, and a perforation device 110, also known as a piercing device configured for piercing target tissue, e.g., the atrial septum 75. In the illustrated embodiment, the perforation device 110 is a radiofrequency (RF) piercing device having a distal end portion 112 terminating in a tip electrode 115. As shown, in the assembled use state illustrated in FIGS. 1A-1C, the perforation device 110 can be disposed within the dilator 105, which itself can be disposed within the sheath 100. In one embodiment in which the transseptal access system 50 is deployed into the right atrium 55 via the IVC 105, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20. When the guidewire, sheath 100, and dilator 105 have been positioned in the superior vena cava, the guidewire is removed from the body, and the sheath 100 and the dilator 105 are retracted so that their distal ends are positioned in the right atrium 55. The perforation device 110 described can then be introduced into the dilator 105, and advanced toward the heart 20.

Subsequently, the user may position the distal end of the dilator 105 against the atrial septum 75, which can be done under imaging guidance. The perforation device 110 is then positioned such that electrode 115 is aligned with or protruding slightly from the distal end of the dilator 105. The dilator 105 and the perforation device 110 may be dragged along the atrial septum 75 and positioned, for example against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode 115 at the desired target site. In addition, tactile feedback provided by medical perforation device 110 is usable to facilitate positioning of the tip electrode 115 at the desired target site.

With the tip electrode 115 and dilator 105 positioned at the target site, energy is delivered from an energy source, e.g., an RF generator, through the RF perforation apparatus 20 to the tip electrode 115 and the target site. In some embodiments, the energy is delivered at a power of at least about 5 W at a voltage of at least about 75 V (peak-to-peak), and functions to vaporize cells in the vicinity of the tip electrode 115, thereby creating a void or perforation through the tissue at the target site. The user then applies force to the perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 s and about 5 s.

With the tip electrode 115 of the perforation device 110 having crossed the atrial septum 75, the dilator 105 can be advanced forward, with the tapered distal tip portion 107 operating to gradually enlarge the perforation to permit advancement of the distal end of the sheath 100 into the left atrium 60.

In some embodiments, the distal end portion 112 of the perforation device 110 may be pre-formed to assume an atraumatic shape such as a J-shape (as shown in FIGS. 1B-1C), a pigtail shape or other shape selected to direct the tip electrode 115 away from the endocardial surfaces of the left atrium 60. Examples of such RF perforation devices can be found, for example, in U.S. patent application Ser. Nos. 16/445,790 and 16/346,404 assigned to Baylis Medical Company, Inc. The aforementioned pre-formed shapes can advantageously function to minimize the risk of unintended contact between the tip electrode 115 and tissue within the left atrium 60 and can also operate to anchor the distal end portion 112 within the left atrium 60 during subsequent procedural steps. For example, in embodiments, the perforation device 110 can be structurally configured to function as a delivery rail for deployment of a relatively larger bore therapy delivery sheath and associated dilator(s). In such embodiments, the dilator 105 and the sheath 100 are withdrawn following deployment of the distal end portion 112 of the perforation device into the left atrium 60. The anchoring function of the pre-formed distal end portion 112 inhibits unintended retraction of the distal end portion 112, and corresponding loss of access to the perforated site on the atrial septum 75, during such withdrawal.

The present disclosure describes novel devices and methods for providing transseptal access to the left atrium 60. In some embodiments, the transseptal access system 50 may be configured to achieve a plurality of different curvatures. This is useful to allow introduction into and positioning of the system 50 at a desired location within the heart 20. For example, the various curvatures allow for achieving desired positioning of the dilator 105 and the perforation device 110 along a portion of the atrial septum 75.

It will be appreciated by the skilled artisan that the perforation device 110 can take on forms other than an RF perforation device. For example, the embodiments of the present disclosure can readily accommodate mechanical perforation devices, e.g., such as a mechanical needle.

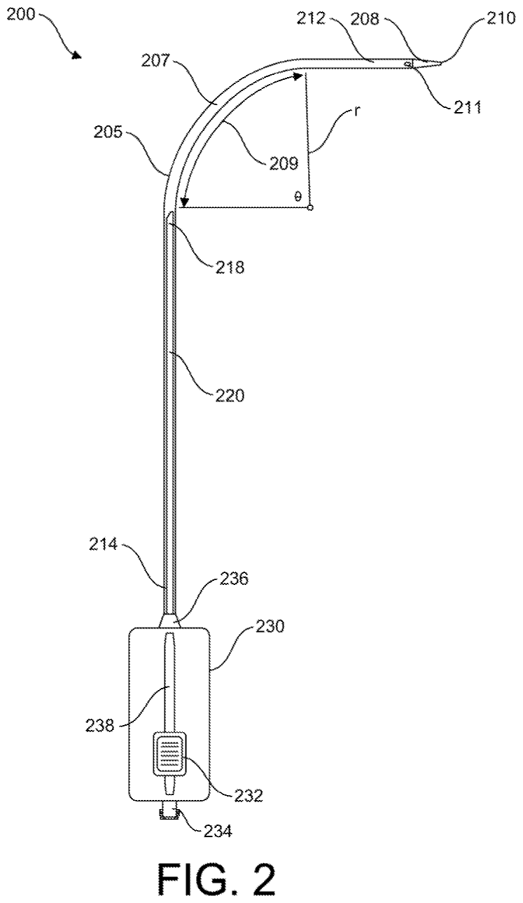

FIG. 2 illustrates an embodiment of a transseptal access system 200 which includes a steerable dilator 205, an inner member 220, and a handle. The dilator 205 has a distal portion 212 including a tapered distal end 212 and a proximal portion 212 including a proximal end. A handle 230 is located at the proximal end of the dilator 205. The handle 230 includes a slider 232 that is used to advance or retract the inner member 220 within the dilator 205.

The dilator 205 is a hollow cannula configured such that it has a pre-formed or preset curve 207 when no force is applied thereto. The pre-formed curve 207 may include a radius of curvature r of in the range of 2 to 5 inches and may include an arc length 209 in the range of 1 to 8 inches. In one aspect, the pre-formed curve 207 places a distal portion 212 of the dilator 205 at approximately 90 degrees from a proximal portion 214 of the dilator 205. The dilator 205 has a bend rigidity lower than that of the inner member 220 such that when the inner member 220 is advanced within the dilator 205 the shape of the dilator 205 changes. The inner member 220 may be a hypotube or other hollow cannula having a lumen. The dilator 205 may have a bend rigidity in the range of 0.01 to 4.62 lbf/in² and a modulus of elasticity in the range of 1×10⁷ to 1×10⁹ Pa. The inner member 220 may have a stiffness in the range of 0.07 to 130.42 lbf/in² and a modulus of elasticity in the range of 3×10⁹ to 5×10¹¹ Pa.

The dilator 205 includes a tapered distal portion 208 and a distal opening 210 through which a guiding device, perforating device, or puncturing device may extend. One or more marker 211 may be located on the dilator 205 to allow for identifying a location of the system 200 during a procedure using an imaging modality. For example, one or more marker 211 may be used to identify the tip or the tapered portion 208 of the dilator. In one aspect, the one or more marker 211 may include barium sulfate integral with the polymer forming the dilator wall.

The system 200 can be packaged as a kit and be ready for use right out of the package. Alternatively, the kit may include a plurality of dilators 205 having different pre-shaped portions 207, and a plurality of inner members having varying degrees of stiffness.

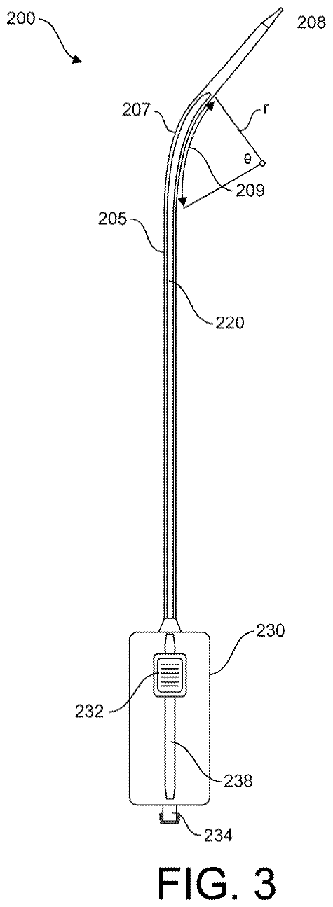

FIG. 3 illustrates the inner member 220 advanced to a most distal position within the dilator 205. In order to place the inner member 220 in the most distal position, the slider 232 is advanced to a most distal position along a track 238 on the handle 230. As shown, the pre-formed curve 207 has been modified as a result of the stiffer inner member 220. While FIG. 3 shows one configuration, it is understood that the inner member 220 may be configured more stiff such that the pre-formed curve 207 takes an entirely straight arrangement, or that the inner member 220 may be configured less stiff such that the pre-formed curve 207 remains more curved.

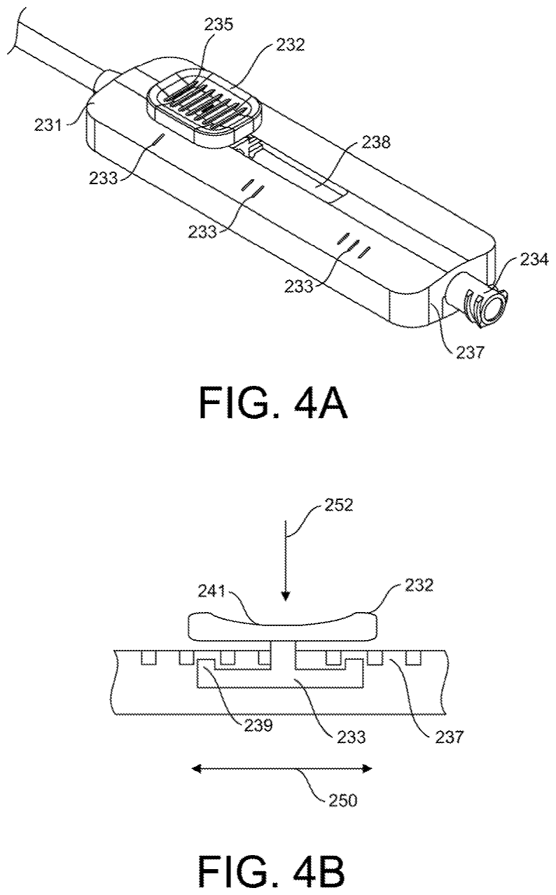

FIG. 4A illustrates the handle 230 for advancing and retracting the inner member 220. The handle 230 may include a substantially rectangular housing having a proximal end 237 and a distal end 231. The distal end 231 includes a connector 236 that is configured to removably attach to the proximal end 214 of the dilator 205. In some aspects, the handle 230 and the dilator may be permanently connected.

The inner member 220 is configured to be translated within the dilator 205 by actuation of the slider 232. The slider 232 moves along a channel or track 238 formed in the handle 230. The length of the channel 238 is at least as long as the arc length 209 of the pre-formed curve 207. In some aspects, the length of the channel 238 is substantially similar to the arc length 209 of the pre-formed curve 207.

The slider 232 includes a surface configured to contact a portion of a user's finger or thumb. In some aspects, the slider 232 may include a textured or knurled surface 235 to increase friction between the slider 232 and the finger or thumb. The slider 232 includes a rectangular shape having a depression 241 into which the finger or thumb may be placed. In other aspects, the slider 232 may have a circular, oval, triangular, square, or other shape as desired.

As illustrated in FIG. 4B, the slider 232 includes a portion 233 that extends into the interior of the handle 230 and is connected to the inner member 220. The inner member 220 may be permanently affixed to the portion 233 or may be removably connected thereto. In some aspects, the portion 233 includes a series of ridges or teeth 239 configured to interact with a plurality of cavities 243 along a portion of the track 238 within the handle 230 in order to allow for stepwise increments in positioning of the slider 232. The slider 232 may be configured to selectively interact with the cavities 243 to set the slider 232 in a desired location along the track 238. This allows for unlocking and locking of the slider 232 in desired portions along the track 238 in order to move the inner member 220 to a desired position within the dilator 205, ultimately achieving a desired shape of the dilator 205.

As shown in FIG. 4B, the slider 232 may be depressed as indicated by arrow 252 and moved along the track as indicated by arrow 25 to retract or advance the inner member 220. Releasing the slider 232 allows the ridges or teeth 239 to seat within the cavities 243, thus locking the slider 232 in place. Locking the inner member 220 in relationship to the dilator 205 allows for a large amount of pressure to be applied to the dilator 205 when the dilator 205 is used to cross the septum 75 without having the inner member 220 slip or change position within the dilator 205.

In some aspects, the series of ridges or teeth 239 interacting with the cavities 243 provides tactile feedback to a user of the system 200. In some aspects, the ridges or teeth 239 may be somewhat flexible, allowing for the slider 232 to move along the track 238 without the need to be depressed, yet being rigid enough that the slider 232 remains in place when the ridges or teeth 239 are within the cavities 243. While not illustrated, the ridges or teeth 239 as well as the cavities may include at least one angled or ramped surface to allow for relative translation to one another easier.

As shown in FIG. 4A, a series of surface markers 233 may be positioned on the handle 230 adjacent the slider 232. The surface markers 233 can be configured to provide a user with an indication of the shape of the dilator 205 as a result of the position of the slider 232, and thus the inner member 220.

The proximal portion 237 of the handle 230 includes a connector 234 configured to allow for selective connection to a desired medical device. In some aspects, the connector 234 may include a luer connector. The connector 234 may allow for introduction of fluids, such as contrast material, into the dilator 205. The connector 234 may also allow for introduction of a guiding member or a perforation or piercing device, such as a radiofrequency piercer, into the dilator 105. The inner member 220 includes a lumen such that a guiding member or piercing device is slidable through the inner member 220 to the distal opening 210 of the dilator 205.

The perforation or piercing device 110 may include a radiofrequency electrode 115 at the distal tip thereof. The piercing device can be connected to a radiofrequency generator, which may in turn be connected to one or more grounding pads, so that radiofrequency energy can be delivered from the radiofrequency generator to the radiofrequency electrode 115. The piercing device 110 may include a core or wire that connects the generator to the electrode 115. When the electrode 115 is in contact with the septum 75, radiofrequency energy may be provided to the electrode 115 to initiate puncturing of the septum 115 as described above. In other aspects, the piercing device 110 may include a sharp tip and be advanced though the septum solely by force.

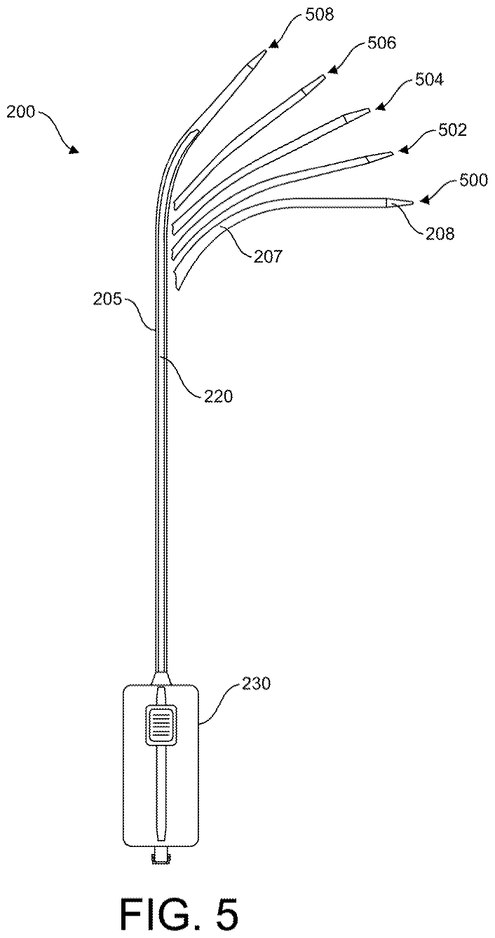

FIG. 5 illustrates a plurality of shapes that can be created in the system 200 by moving the inner member 220 between a most proximal position and a most distal position within the dilator 205. When the slider 232 of the handle is placed in a most proximal position, the inner member is withdrawn entirely from the pre-formed portion 207 of the dilator 205. This allows the dilator 205 to achieve its pre-formed curvature which is illustrated at 500 in FIG. 5. When the slider 232 is in the most proximal position, the distal tapered portion 208 of the dilator may be positioned at approximately 90 degrees from the proximal portion 214 of the dilator 205. In some aspects, the distal tapered portion 208 of the dilator may be positioned at greater than 90 degrees in relationship to the proximal portion 214 or at less than 90 degrees in relationship to the proximal portion 214.

When the slider 232 of the handle is placed in a most distal position, the inner member 220 achieves its most distal position within the dilator 205. In this configuration, the stiffness of the inner member 220 reduces the curve 207 of the dilator 205 such that the dilator 205 achieves its most linear configuration as illustrated at 508. It is understood that the shape of the curve can be adjusted based on the stiffness of the inner member 220. For example, in some aspects when the slider 232 is positioned in the most distal position, a relatively stiff inner member 220 may entirely overcome the force provided by the pre-formed curve 207 such that the dilator 205 is substantially straight. To the contrary, a relatively flexible inner member 220 would result in the dilator 205 having a more pronounced curve when the slider 232 is in the most distal position.

FIG. 5 illustrates various shapes 502, 504, 506 of the dilator when the slider of the handle is positioned between the most distal and most proximal positions. While only a limited number of shapes are illustrated, it is understood that the dilator 205 can achieve a multitude of curvatures based on how much the inner member 220 is retracted from the dilator 205.

In some aspects, the dilator 205 and/or the inner member 220 may be formed of a polymeric or metallic material, or a combination thereof. In embodiments, the inner member 220 may be formed of a material having superelastic and/or shape memory characteristics. One exemplary suitable metallic material is a nickel titanium (NiTi) alloy. The body of the dilator 205 and/or the inner member 220 may include regions having different stiffness or be formed of different materials. For example, a proximal portion may be formed of a first stiffer material while a distal portion may be formed by a second more flexible material.

In some aspects, the inner member 220 may also include one or more markers along a portion thereof for identifying a position or location of the inner member while using imaging. In some aspects, the dilator 205 and/or the inner member 220 may include a stiffening member such as a braid or coil. In some aspects, only portions of the dilator 205 and/or the inner member 220 may include a stiffening member, such as only the proximal portion.

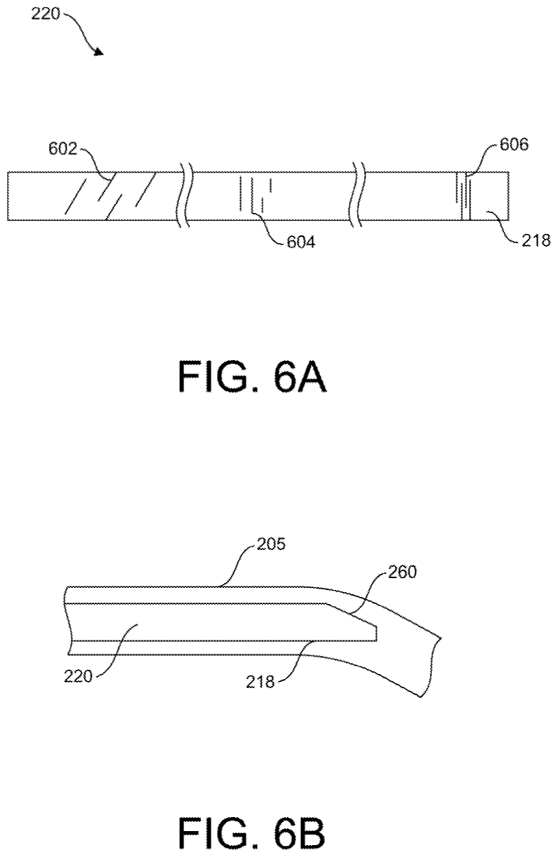

FIG. 6A illustrates an example configuration for an inner member 220 in accordance with one embodiment of the disclosure. The body of the inner member 220 has a plurality of cuts 602, 604, 606 machined into the wall, for example by laser cutting. The shape and positioning of the cuts allows for a transition in flexibility from the proximal portion of the inner member to the distal portion 218. The cuts may include a broken spiral configuration as shown at 602 or may be positioned substantially orthogonal to the longitudinal axis as shown at 604 and 606. In some aspects, there may be a single cut that winds around the axis with a wider spacing between loops at the proximal portion and a larger spacing at the distal portion. The spacing and size of the cuts can be varied to achieve different flexibilities along the inner member 220.

In some aspects, the distal tip portion 218 of the inner member 220 may be sliced or otherwise shaped so that the distal portion includes bevel 260 as illustrated in FIG. 6B. This allows the tip of the inner member 220 to more easily move along the inner surface of the dilator 205 in the pre-shaped region. In other embodiments, the distal end of the inner member may be rounded and polished smooth to prevent any portion of the inner member from piercing or gouging the wall of the dilator 205.

In some aspects, the inner member 220 may be formed of a shape memory material, such as a shape memory polymer or a shape memory metal. In this configuration, the inner member 220 may have a first shape at a first temperature, and a second shape at a second temperature. By providing the inner member 220 with its own curvature, more shapes of the dilator 205 can be achieved. Shape transition may be initiated in the inner member 220 by inserting a heated solution into the dilator 205 or using electricity to heat a portion of the inner member 220.

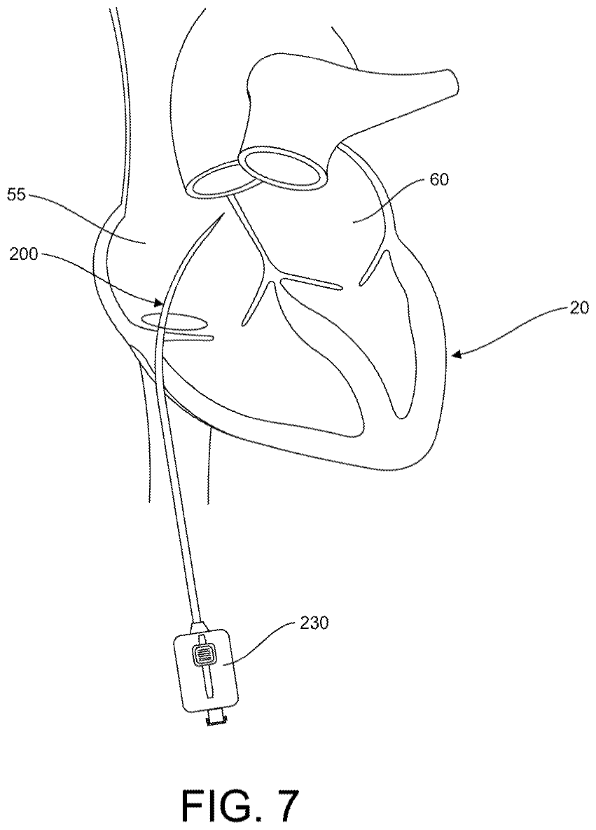

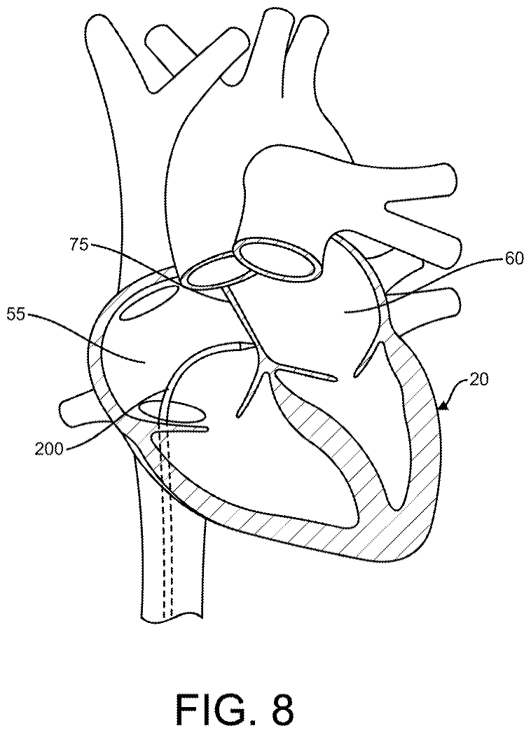

FIG. 7 illustrates the system 200 in a first configuration during a portion of a transseptal access procedure. As illustrated in FIG. 7, the system is introduced into a patient's heart 20 through the inferior vena cava (IVC) 85. During insertion, the slider 232 of the handle 230 may be placed in the most distal or near the most distal position in order to significantly straighten the pre-formed curve 237 of the dilator 205 by way of advancement of the inner member 220. Once the distal portion of the dilator 205 is located inside of the right atrium 55, a user may change the shape of the dilator 205 as illustrated in FIG. 8 to position the tip of the dilator 205 at a desired location along the septum 75.

FIG. 8 illustrates the system 200 in a second configuration during a portion of a transseptal access procedure. During this portion of the procedure, a user has withdrawn the inner member 220 from the dilator 205 allowing the pre-formed portion 207 of the dilator 205 to achieve its unobstructed shape. In this configuration, the distal tip of the dilator 205 is placed against the septum 75 and a piercing device 110 may be inserted through the connector 234 of the handle 230 and guided to the distal tip of the dilator 205 to pierce the septum 75.

While FIG. 7 and FIG. 8 illustrate the inner member 220 being in the most proximal and most distal positions, respectively, it is understood that the inner member 220 may be located in various locations within the dilator 205 for different portions of the procedure. By providing the dilator 205 with the ability to achieve a multitude of curvatures, a user is able to adapt the system 200 for use in patient's having a variety of heart sizes.

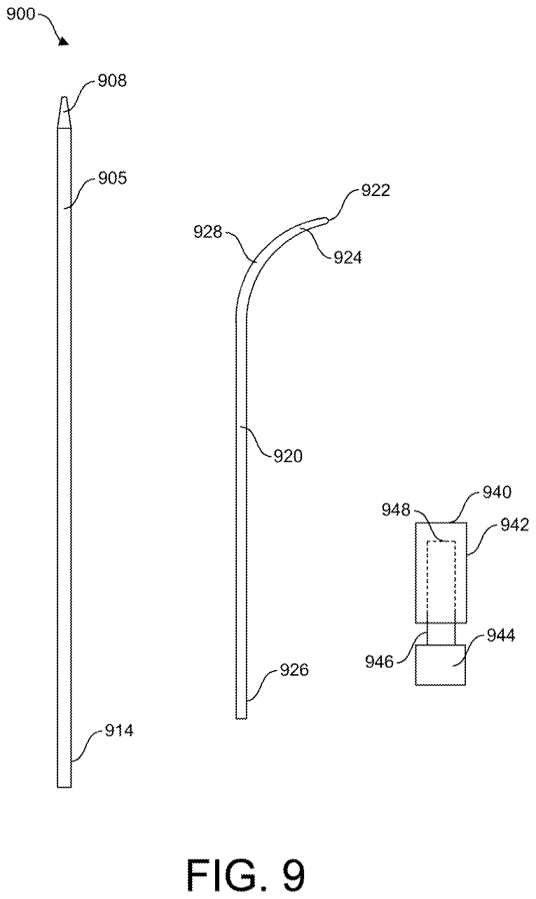

FIG. 9 illustrates an exploded view of another embodiment of a transseptal access system 900. While similar to the system discussed above, system 900 includes a dilator 905 having a substantially straight configuration and an inner member 920 that includes a pre-formed curve 928 or a region that transitions into a curve via a shape-changing material. Like the system described above, advancing and retracting the inner member 920 within the dilator 905 will vary the overall shape of the system 900.

The distal end 924 of the inner member 920 ends in a rounded or polished distal tip 922 having an opening to receive a guiding or piercing member. The proximal end 926 of the inner member 920 removably connects to the distal end 948 of a plunger 946 of a handle 942. The handle distal end 940 is configured to removably connect to the proximal end 914 of the dilator 905. The plunger 946 includes a hub 944 that can be grasped by a user to translate the plunger 946 into and out of the handle 942, thus extending and retracting the inner member 920 into and out of the dilator 905.

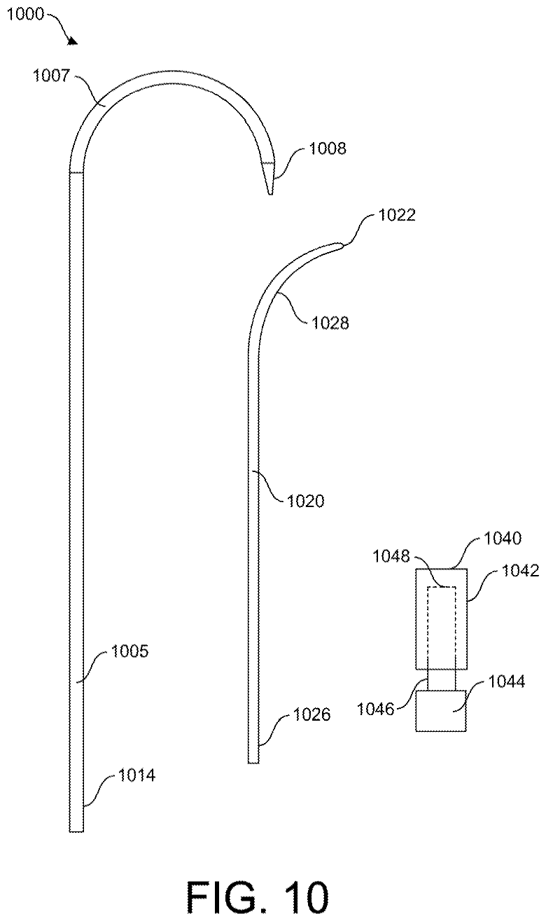

FIG. 10 illustrates another embodiment for a transseptal access system 1000. In this embodiment, both the dilator 1005 and inner member 102 include a pre-formed curved region 1007 and 1028 respectively. Because both the dilator 1005 and the inner member 1020 include pre-formed curves, more complex shapes can be formed by translating the inner member 1020 within the dilator 1005.

Like system 900, the distal end of the inner member 1020 ends in a rounded or polished distal tip 1022 having an opening to receive a guiding or piercing member. The proximal end 1026 of the inner member 1020 removably connects to the distal end 1048 of a plunger 1046 that is configured to translate inside of a handle 1042. The handle distal end 1040 is configured to removably connect to the proximal end 1014 of the dilator 1005. The plunger 1046 includes a hub 1044 that can be grasped by a user to translate the plunger 1046 into and out of the handle 1042, thus extending and retracting the inner member 1020 into and out of the dilator 1005.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system for use in a transseptal crossing, comprising:
an outer cannula having a proximal end, a tapered distal end, a preset curve, and a proximal portion extending between the proximal end and the preset curve, wherein the preset curve is configured to position the tapered distal end at approximately 90 degrees relative to the proximal portion;
an inner member configured to slide within the outer cannula between a first position and a second position, the inner member having a stiffness greater than a stiffness of the outer cannula, wherein in the first position the tapered distal end is positioned approximately 90 degrees relative to the proximal portion and in the second position the tapered distal end is positioned greater than 90 degrees relative to the proximal portion; and
a control mechanism having a first portion connected to the proximal end of the outer cannula and a second portion connected to a proximal end of the inner member, wherein the control mechanism is configured to move the inner member between the first position and the second position.

2. The system of claim 1, further comprising a piercing member.

3. The system of claim 2, wherein the piercing member includes a distal radiofrequency electrode.

4. The system of claim 1, wherein the control mechanism comprises a handle.

5. The system of claim 1, wherein the control mechanism comprises a slider.

6. The system of claim 5, wherein the slider forms part of a ratcheting system configured to lock the inner member in a desired position within the outer cannula.

7. The system of claim 5, wherein the slider moves along a track.

8. The system of claim 7, wherein the track has a length that is substantially the same as an arc length of the preset curve.

9. The system of claim 1, wherein the outer cannula is formed of one or more polymer and metallic material.

10. The system of claim 1, further comprising one or more marker located on a portion of the outer cannula or the inner member.

11. The system of claim 10, wherein the one or more marker is radiopaque.

12. The system of claim 1, wherein the outer cannula or the inner member includes a reinforcing material.

13. The system of claim 1, wherein the outer cannula has a bend rigidity in the range of 0.01 to 4.62 lbf/in² and a modulus of elasticity in the range of 1×10⁷ to 1×10⁹ Pa.

14. The system of claim 1, wherein the inner member has a bend rigidity in the range of 0.07 to 130.42 lbf/in² and a modulus of elasticity in the range of 3×10⁹ to 5×10¹¹ Pa.

15. The system of claim 1, wherein the inner member includes a beveled or polished distal tip.

16. The system of claim 1, wherein the inner member includes one or more slot or groove and increases in flexibility from a proximal end to a distal end.

17. A dilator for use in a transseptal crossing, comprising:
an outer cannula having a proximal end, a tapered distal end, a preset curve, and a proximal portion extending between the proximal end and the preset curve;
an inner member configured to slide within the outer cannula between a first position and a second position, the inner member having a stiffness greater than a stiffness of the outer cannula, wherein in the first position the tapered distal end is positioned approximately 90 degrees relative to the proximal portion and in the second position the tapered distal end is positioned greater than 90 degrees relative to the proximal portion; and
a control mechanism having a first portion connected to the proximal end of the outer cannula and a second portion connected to a proximal end of the inner member, wherein the control mechanism is configured to move the inner member between the first position and the second position.

18. The dilator of claim 17, wherein the preset curve has an arc length in the range of 1 to 8 inches.

19. The dilator of claim 17, wherein the preset curve has a radius of curvature in the range of 2 to 5 inches.

20. A method for performing a transseptal procedure, comprising:
positioning an outer cannula having an inner stiffening member into a right atrium of a patient;
withdrawing the inner stiffening member from a portion of the outer cannula to change a shape of the outer cannula;
placing a distal end of the outer cannula on the transseptal septum;
advancing a piercing member though the inner stiffening member and the outer cannula; and
puncturing the transseptal septum with the piercing member.
